(19) United States
(12) Patent Application Publication
(10) Pub. No.: US 2025/0243227 A1
(43) Pub. Date: Jul. 31, 2025

(54) IDURONIDASE STABILIZERS AND USES THEREOF

(71) Applicant: Academia Sinica, Taipei (TW)

(72) Inventors: Wei-Chieh CHENG, Taipei City (TW); Shih-Ying CHANG, Yunlin County (TW); Hsuan-Hsuan TENG, New Taipei City (TW); Hsi-Ju WU, Tainan City (TW); Hung-Yi LIN, New Taipei City (TW)

(21) Appl. No.: 18/855,314

(22) PCT Filed: Apr. 10, 2023
(86) PCT No.: PCT/US2023/017994

§ 371 Date: Oct. 9, 2024

Related U.S. Application Data
(60) Provisional application No. 63/330,783, filed on Apr. 14, 2022.

(51) Int. Cl. C07F 9/59; A61K 31/665; A61K 31/675; A61K 38/47; C07F 9/655

(52) U.S. Cl. CPC C07F 9/59; A61K 31/665; A61K 31/675; A61K 38/47; C07F 9/6552; C12Y 302/01076

(57) ABSTRACT

Disclosed herein are compounds that stabilize recombinant human α-iduronidase (rh-α-IDUA) activity, and their uses in the treatment and/or prophylaxis of lysosomal storage diseases (LSDs), such as mucopolysaccharidosis type I (MPS1). The compound disclosed herein has the structure of formula (I),

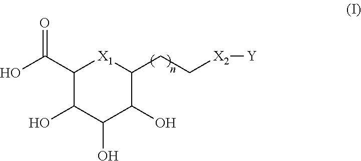

wherein, n is an integral between 0 and 2; X₁ is O or —NH; X₂ is O, —NH or —NR^{a}, in
which R^{a} is C₁₋₁₀alkyl; Y is H or

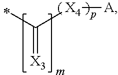

wherein m and p are independently 0 or 1; X₃ is S, O, or —NH; X₄ is O, —NH, methylene, or —CH₂(C₁₋₁₀)alkyl; and A is aryl, heteroaryl, or heterocyclyl optionally substituted with one or more substituent selected from the group consisting of halo, hydroxyl, amino and phosphate.

### CROSS REFERENCES TO RELATED APPLICATION

This application is a U.S. National Stage Filing under 35 U.S.C from International Patent Application Serial No. PCT/US2023/17994, filed Apr. 10, 2023, and published on Oct. 19, 2023, which claims priority and the benefit of U.S. Provisional Patent Application No. 63/330,783, filed Apr. 14, 2022, the entirety of which is incorporated herein by reference.

### BACKGROUND OF THE INVENTION

### 1. Field of the Invention

The present disclosure in general relates to the field of therapeutics for a mucopolysaccharidosis; more particularly to novel compounds that stabilize recombinant human α-iduronidase (rh-α-IDUA), and their uses in the treatment and/or mucopolysaccharidosis type I (MPS1).

### 2. Description of Related Art

The mucopolysaccharidoses (MPS) are degenerative genetic diseases caused by deficiency of enzymes which catalyze the degradation of specific glycosaminoglycans (GAGs) known as dermatan sulfate (DS) and heparan sulfate (HS). GAGs contain long unbranched polysaccharides characterized by a repeating disaccharide unit. The enzymatic deficiency caused an accumulation of GAGs in the cells, the tissues, and in particularly, the cell lysosomes of affected subjects, leading to permanent and progressive cell damage, which affects the appearance, the physical capacities, the organ function and, in most cases, the metal development of affected subjects.

The several mucopolysaccharidoses are distinguished by the particular enzyme affected in GAG degradation. For example, mucopolysaccharidosis type I (MPS1) is caused by a deficiency of α-L-iduronidase (α-IDUA), which hydrolyzes the terminal α-iduronic acid residues of dermatan sulfate. Clinical presentation of MPS1 correlates with the degree of residual enzyme activity. Several approaches have been used for the treatment of MSP, most of which focus on enzyme replacement therapy (ERT) or gene therapy for use in disease management. ERT involves administration of an exogenously-produced natural or recombinant enzyme (e.g., recombinant human α-iduronidase (rh-α-IDUA)); while the gene therapy involves administration of a vector that express a recombinant enzyme in the subject. However, patients receive supplemented enzyme either through ERT or gene therapy tend to develop antibodies against the replacement enzyme after long term therapy.

Accordingly, there exist in this art an improved method for the treatment and/or prophylaxis of diseases and/or disorders associated with the accumulation of HS, particularly, MPS1.

### SUMMARY

Inventors of the present disclosure designed and synthesized novel iduronic acid/iminoiduronic acid-based compounds that stabilized α-L-iduronidase (IDUA) and mitigated environmental stressors experienced by the enzyme, which improved hydrolysis of the terminal α-L-iduronic acid residue in glycosaminoglycans (e.g., heparan sulfate (HS)). Thus, the newly produced compounds are potential candidates for the development of medicaments suitable for the treatment and/or prophylaxis of diseases and/or disorders associated with the accumulation of HS, such as mucopolysaccharidosis type I (MPS1).

Accordingly, one aspect of the present disclosure is to provide a novel compound having the structure of formula (I), a pharmaceutically acceptable salt, a solvate or a stereoisomer thereof,

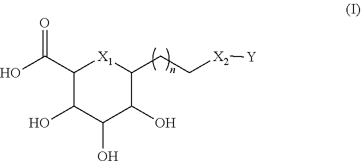

wherein,
n is an integral between 0 and 2;
X₁ is O or —NH;
X₂ is O, —NH or —NR^{a}, in which R^{a} is C₁₋₁₀alkyl;
Y is H or

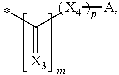

in which
   m and p are independently 0 or 1;
   X₃ is S, O, or —NH;
   X₄ is O, —NH, methylene, or —CH₂(C₁₋₁₀)alkyl; and
   A is aryl, heteroaryl, or heterocyclyl optionally substituted with one or more substituent selected from the group consisting of halo, hydroxyl, amino and phosphate.

According to one preferred embodiment of the present disclosure, in the compound of formula (I), n is 1, X₁ is O, X₂ is —NH, and Y is H.

According to other embodiments of the present disclosure, particular compounds are of formula (II),

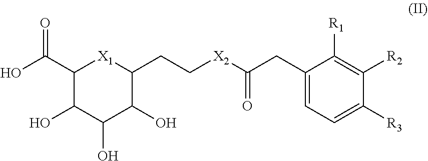

wherein,
X₁ is O or —NH;
X₂ is O, —NH or —NR^{a}, in which R^{a} is C₁₋₁₀alkyl; and
R₁, R₂, and R₃ are independently H or phosphate.

According to some embodiments, the compound of formula (II) is selected from the group consisting of,

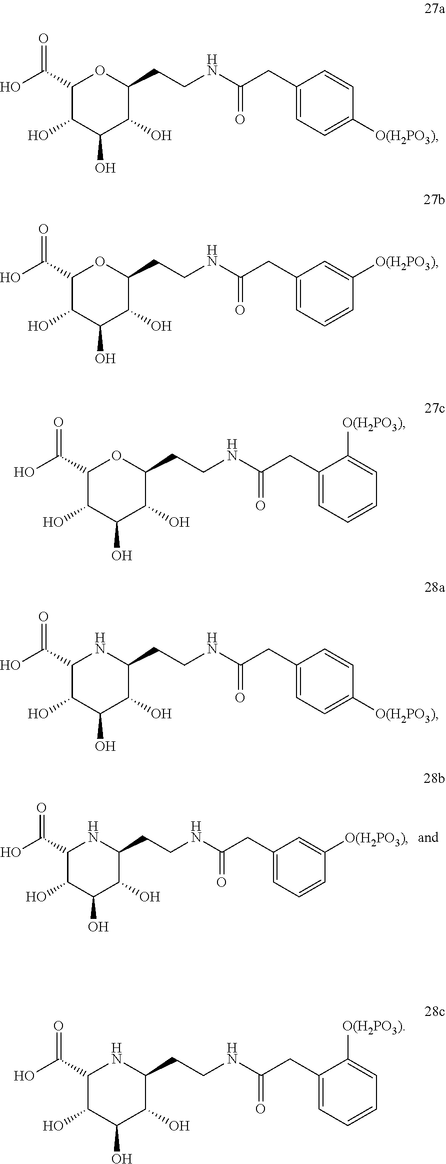

According to one preferred embodiment of the present disclosure, the compound of formula (II) is

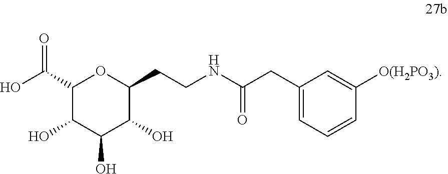

The second aspect of the present disclosure is to provide a pharmaceutical composition for the treatment or prophylaxis of a subject having or suspected of having mucopolysaccharidosis type I (MPS I). The pharmaceutical composition comprises a therapeutically or prophylactically effective amount of the compound of formula (I); and a pharmaceutically acceptable carrier.

The compound of formula (I) is present at a level of about 0.1% to 99% by weight, based on the total weight of the pharmaceutical composition. In some embodiments, the compound of formula (I) is present at a level of at least 1% by weight, based on the total weight of the pharmaceutical composition. In certain embodiments, the compound of formula (I) is present at a level of at least 5% by weight, based on the total weight of the pharmaceutical composition. In still other embodiments, the compound of formula (I) is present at a level of at least 10% by weight, based on the total weight of the pharmaceutical composition. In still yet other embodiments, the compound of formula (I) is present at a level of at least 25% by weight, based on the total weight of the pharmaceutical composition.

Preferably, in the compound of formula (I), n is 1, X₁ is O, X₂ is —NH, and Y is H.

Optionally or alternatively, the compound has the structure of formula (II)

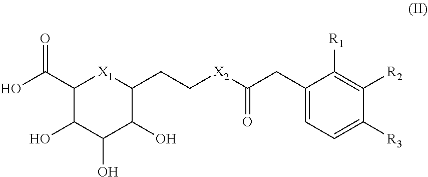

wherein,
X₁ is O or —NH;
X₂ is O, —NH or —NR^{a}, in which R^{a} is C₁₋₁₀alkyl; and
R₁, R₂, and R₃ are independently H or phosphate.

According to some preferred embodiments, the compound of formula (II) is selected from the group consisting of,

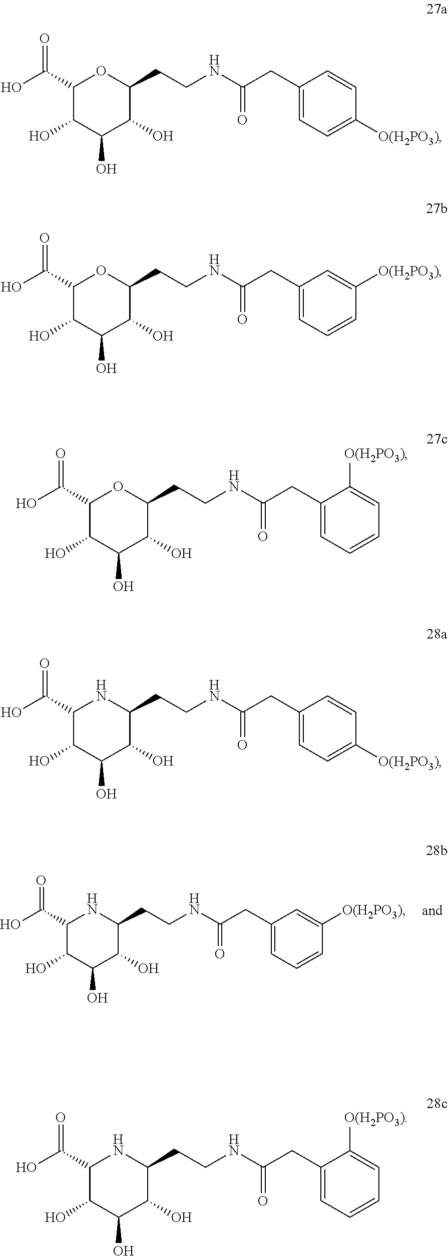

Preferably, the compound of formula (II) is

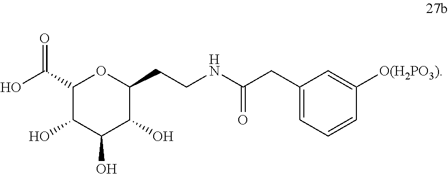

The present disclosure also encompasses a method for the treatment or prophylaxis of a subject having or suspected of having a mucopolysaccharidosis type I. The method comprises the step of administering to the subject a recombinant human α-Iduronidas (rh-α-IDUA) before, together with, or after the administration of present pharmaceutical composition.

The present disclosure also encompasses kits useful for the treatment or prophylaxis of a subject having a mucopolysaccharidosis type I. The kit includes, at least, a first container containing the compound of formula (I); and a second container containing a rh-α-IDUA.

The details of one or more embodiments of this disclosure are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The accompanying drawings, which are incorporated in and constitute a part of the specification, illustrate various example systems, methods and other exemplified embodiments of various aspects of the invention. The present description will be better understood from the following detailed description read in light of the accompanying drawings, where,

FIG. 1. The cell-based rh-α-IDUA stabilization study of the compounds of Example 1/rh-α-IDUA in GM01391 fibroblasts. (A) Preliminary cell-based screening of compounds of Example 1 by the co-treatment of rh-α-IDUA (0.1 nM) with 12 or 27b (100 M); (B) A dose-dependent experiment for 12, 27a, or 27b with 1 nM of rh-α-IDUA; (C) Co-treatment of 12 and rh-α-IDUA (1 or 0.1 nM) on IDUA activity; and (D) 12/rh-α-IDUA co-treatment on HS reduction in cells. Each bar represents the mean±SD of three determinations. *P<0.05; **P<0.01; ***P<0.001 compared to rh-α-IDUA alone, as determined by one-way ANOVA with Dunnett post-hoc analysis.

FIG. 2. The cell-based rh-α-IDUA stabilization study of 12/rh-α-IDUA in GM02846 fibroblasts. (A) IDUA activity, and (B) the reduction of HS; Each bar represents the mean±SD of three determinations. *P<0.05; **P<0.01; ***P<0.001 compared to rh-α-IDUA alone, as determined by one-way ANOVA with Dunnett post-hoc analysis.

### DETAILED DESCRIPTION OF THE PREFERRED EMBODIMENTS

The detailed description provided below in connection with the appended drawings is intended as a description of the present disclosure and is not intended to represent the only forms in which the present disclosure may be constructed or utilized.

### 1. Definitions

Unless otherwise indicated, the term “substituted,” when used to describe a chemical structure or moiety, refers to a derivative of that structure or moiety wherein one or more of its hydrogen atoms is substituted with an atom, chemical moiety or functional group such as, but not limited to, —OH, —O(H₂PO₃), alkyl (e.g., methyl, ethyl, propyl, t-butyl), alkoxy, alkanoyloxy (e.g., —OAc), alkenyl, aryl, aryloxy, halo, or haloalkyl.

“Heterocyclyl” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. Heterocyclyl includes heteroaryl. Heterocyclyl also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Halo” refers to fluorine (fluoro, —F), chlorine (chloro, —Cl), bromine (bromo, —Br), or iodine (iodo, —I).

An atom, moiety, or group described herein may be unsubstituted or substituted, as valency permits, unless otherwise provided expressly. The term “optionally substituted” refers to substituted or unsubstituted.

The present disclosure is not intended to be limited in any manner by the above exemplary listing of substituents.

The term “salt” refers herein as a salt which is formed by the interaction of an acid (e.g., the present compound) with a base, including organic or inorganic types of bases, such as sodium hydroxide, potassium hydroxide, amine, alkylamine and etc.

The term “solvate” refers to forms of the compound that are associated with a solvent, usually by a solvolysis reaction. This physical association may include hydrogen bonding. Conventional solvents include water, methanol, ethanol, acetic acid, dimethyl sulfoxide (DMSO), tetrahydrofuran (THF), diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates.

It should also be noted that if the stereochemistry of a structure or a portion of a structure is not indicated with, for example, bold or dashed lines, the structure or the portion of the structure is to be interpreted as encompassing all stereoisomers of it. Similarly, names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound is an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, a “prophylactically effective amount” of a compound is an amount sufficient to prevent a disease or condition, or one or more symptoms associated with the disease or condition, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, the terms “treat,” “treating” and “treatment” contemplate an action that occurs while a patient is suffering from the specified disease or disorder, which reduces the severity of the disease or disorder, or one or more of its symptoms, or retards or slows the progression of the disease or disorder.

The term “subject” or “patient” is used interchangeably herein and is intended to mean a mammal including the human species that is susceptible to infection by a virus. The term “mammal” refers to all members of the class Mammalia, including humans, primates, domestic and farm animals, such as rabbit, pig, sheep, and cattle; as well as zoo, sports or pet animals; and rodents, such as mouse and rat. Further, the term “subject” or “patient” intended to refer to both the male and female gender unless one gender is specifically indicated. Accordingly, the term “subject” or “patient” comprises any mammal which may benefit from the treatment method of the present disclosure. Examples of a “subject” or “patient” include, but are not limited to, a human, rat, mouse, guinea pig, monkey, pig, goat, cow, horse, dog, cat, bird, and fowl. In a preferred embodiment, the subject is a human.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a”, “and”, and “the” are used herein to include plural referents unless the context clearly dictates otherwise.

### 2. Novel Iduronidase Stabilizers

This invention encompasses compounds of formula (I), a pharmaceutically acceptable salt, solvate or stereoisomer thereof,

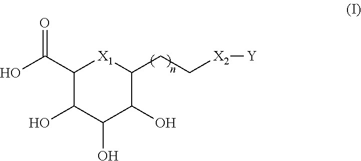

wherein,
n is an integral between 0 and 2;
X₁ is O or —NH;
X₂ is O, —NH or —NR^{a}, in which R^{a} is C₁₋₁₀alkyl;
Y is H or

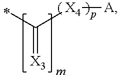

in which
   m and p are independently 0 or 1;
   X₃ is S, O, or —NH;
   X₄ is O, —NH, methylene, or —CH₂(C₁₋₁₀)alkyl; and
   A is aryl, heteroaryl, or heterocyclyl optionally substituted with one or more substituent selected from the group consisting of halo, hydroxyl, amino and phosphate.

According to one preferred embodiment of the present disclosure, in the formula (I), n is 1, X₁ is O, X₂ is —NH, and Y is H.

According to some embodiments of the present disclosure, particular compounds are of formula (II),

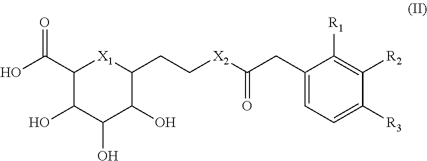

wherein,
X₁ is O or —NH;
X₂ is O, —NH or —NR^{a}, in which R^{a} is C₁₋₁₀alkyl; and
R₁, R₂, and R₃ are independently H or phosphate.

Exemplary compounds of formula (II) include, but not limited to, the followings,

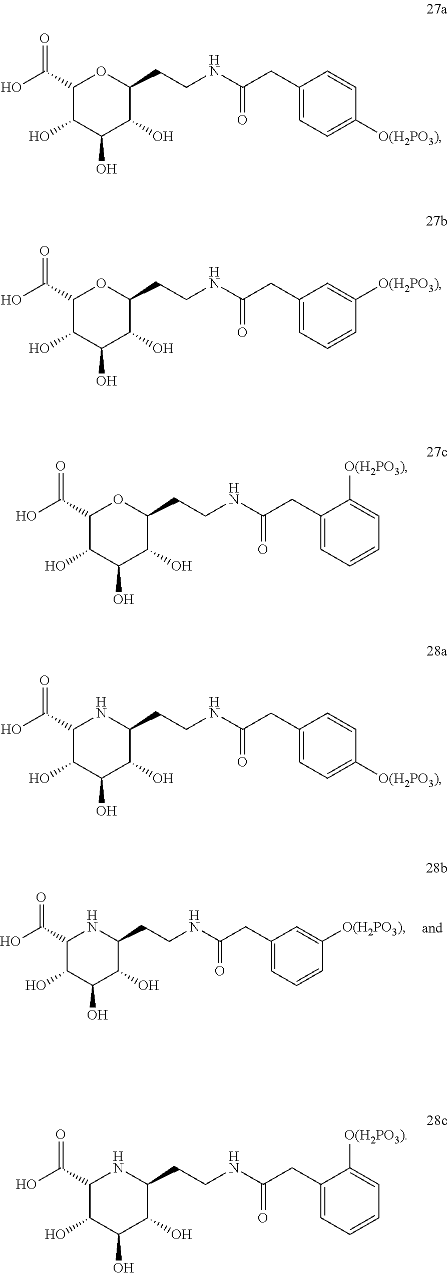

According to one preferred embodiment of the present disclosure, the compound of formula (II) is

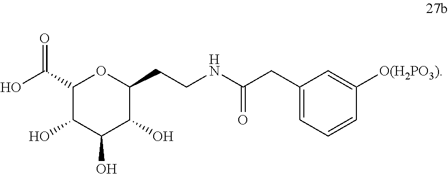

Compounds of the invention contain one or more stereocenters, thus can exist as racemic mixtures of enantiomers or mixtures of diastereomers. This invention thus encompasses stereomerically pure forms of such compounds, as well as mixtures of those forms. Stereoisomers may be asymmetrically synthesized or resolved using standard techniques such as crystallization, chromatography, and the use of a resolving agent. One preferred way of separating enantiomers from a racemic mixture is by use of preparative high performance liquid chromatography (HPLC). Alternatively, the racemic may be separated into its enantiomers by reacting with an optically active form of a resolving agent in the presence of a solvent. Depending on the optical form of the resolving agent, one of the two enantiomers is separated out as an insoluble salt with high yield and high optical purity, while the opposite enantiomer remains in the solution.

The present invention thus further encompasses stereoisomeric mixtures of compounds disclosed herein. It also encompasses configurational isomers of compounds disclosed herein (e.g., cis- and trans-isomers, whether or not involving double bonds), either in admixture or in pure or substantially pure form.

### 3. Method of Use

The present invention encompasses a method for the treatment or prophylaxis of a subject having a mucopolysaccharidosis type I (MPSI). The method comprises the step of administering a therapeutically or prophylactically effective amount of the present compound of formula (I) together with, before or after the administration of a recombinant human α-Iduronidas (rh-α-IDUA) to the subject, so as to reduce the accumulation of heparan sulfate in the subject.

Advantageously, the compound of formula (I) is administered in an amount of 0.01 mg to 5000 mg per day, such as 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000 mg. More preferably, the compound of formula (I) is administered in an amount of 0.1 mg to 2500 mg per day, such as 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500 mg. Most preferably, the compound of formula (I) is administered in an amount of 1 mg to 1000 mg per day, such as 1.0, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 890, 900, 910, 920, 930, 940, 950, 960, 970, 980, 990, 1000 mg.

The compound of formula (I) may be administered one or more times per day, such as twice, or thrice per day, by dividing the daily dose mentioned above for two- or three-times administration. For example, a daily dose of 1000 mg will be administered in a proportion of two doses of 500 mg each. It is understood that each dose may consist of one or more pharmaceutical forms, for example, a dose of 500 mg may consist of two pharmaceutical forms of 250 mg each.

The amount, route of administration and dosing schedule of the compound of formula (I) will depend upon factors such as the specific indication to be treated, prevented, or managed, and the age, sex and condition of the patient. The roles played by such factors are well known in the art, and may be accommodated by routine experimentation.

According to one preferred embodiment, in the formula (I), X is O, and Y is H; and co-administration of the compound and rh-α-IDUA results in enhanced IDUA activity and reduced level of HS in the subject.

### 4. Pharmaceutical Formulation

The present disclosure also encompasses pharmaceutical compositions suitable for use with a recombinant human α-IDUA for the treatment and/or prophylaxis of MPS1.

In some embodiments, the present compound of formula (I) is formulated with one or more pharmaceutically acceptable excipients to form a pharmaceutical composition according to techniques known to those skilled in the art. The compound of formula (I) is present at a level of about 0.1% to 99% by weight, based on the total weight of the pharmaceutical composition. In some embodiments, the compound of formula (I) is present at a level of at least 1% by weight, based on the total weight of the pharmaceutical composition. In certain embodiments, the compound of formula (I) is present at a level of at least 5% by weight, based on the total weight of the pharmaceutical composition. In still other embodiments, the compound of formula (I) is present at a level of at least 10% by weight, based on the total weight of the pharmaceutical composition. In still yet other embodiments, the compound of formula (I) is present at a level of at least 25% by weight, based on the total weight of the pharmaceutical composition.

Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intra-arterial), or transdermal administration to a patient. Examples of dosage forms include, but are not limited to, tablets; caplets; capsules (e.g., soft elastic gelatin capsules); cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration requires enteric coatings to protect the compounds of this invention from degradation within the gastrointestinal tract. Similarly, a formulation may contain ingredients that facilitate delivery of the active ingredient(s) to the site of action. For example, compounds may be administered in liposomal formulations, in order to protect them from degradative enzymes, facilitate transport in circulatory system, and effect delivery across cell membranes to intracellular sites.

Similarly, poorly soluble compounds may be incorporated into liquid dosage forms (and dosage forms suitable for reconstitution) with the aid of solubilizing agents, emulsifiers and surfactants such as, but not limited to, cyclodextrins (e.g., α-cyclodextrin or β-cyclodextrin), and non-aqueous solvents, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, dimethyl sulfoxide (DMSO), biocompatible oils (e.g., cottonseed, groundnut, corn, germ, olive, castor, and sesame oils), glycerol, tetrahydrofurfuryl alcohol, polyethylene glycols, fatty acid esters of sorbitan, and mixtures thereof (e.g., DMSO:corn oil), lipids such as egg york phosphatidylcoline (EPC), soybean phosphatidylcholine (SPC), 1,2-dioleoyl-sn-glycero-3-phosphocholine (DOPC), 1,2-distearoyl-sn-glycero-3-phosphocholine (DSPC), cholesterol (CHO), dipalmitoylphosphatidylcholine (DPPC) and PEG-2000. According to one preferred embodiment, the compound of formula (I) is incorporated into lipids to form liposomes suitable for oral or parenteral administration.

The composition, shape, and type of a dosage form will vary depending on its use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art.

### 4.1 Oral Dosage Forms

Pharmaceutical compositions of the present invention suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art.

Typical oral dosage forms are prepared by combining the active ingredient(s) in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms. If desired, tablets can be coated by standard aqueous or non-aqueous techniques. Such dosage forms can be prepared by conventional methods of pharmacy. In general, pharmaceutical compositions and dosage forms are prepared by uniformly and intimately admixing the active ingredients with liquid carriers, finely divided solid carriers, or both, and then shaping the product into the desired presentation if necessary. Dis-integrants may be incorporated in solid dosage forms to facility rapid dissolution. Lubricants may also be incorporated to facilitate the manufacture of dosage forms (e.g., tablets).

### 4.2 Parenteral Dosage Forms

Parenteral dosage forms can be administered to patients by various routes including, but not limited to, subcutaneous, intravenous (including bolus injection), intramuscular, and intra-arterial. Because their administration typically bypasses patients' natural defenses against contaminants, parenteral dosage forms are specifically sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: water; aqueous vehicles such as, but not limited to, sodium chloride solution, Ringer's solution, and Dextrose; water-miscible vehicles such as, but not limited to, ethyl alcohol, polyethylene glycol, and polypropylene glycol; and non-aqueous vehicles such as, but not limited to, lipids, corn oil, cottonseed oil, peanut oil, sesame oil, ethyl oleate, isopropyl myristate, and benzyl benzoate.

### 4.3 Transdermal, Topical and Mucosal Dosage Forms

Transdermal, topical, and mucosal dosage forms include, but are not limited to, ophthalmic solutions, sprays, aerosols, creams, lotions, ointments, gels, solutions, emulsions, suspensions, or other forms known to one of skill in the art. Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients of the invention. For example, penetration enhancers may be used to assist in delivering active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates may also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition

### 5. Kits

Also encompasses within the present disclosure are kits useful for treatment or prophylaxis of MPS1 in a subject.

The kit according to present disclosure include, at least, a first container containing the present compound of formula (I); a second container containing a recombinant human α-IDUA; and a legend associated with the kit for instructing a user how to use the kit. The legend may be in a form of pamphlet, tape, CD, VCD or DVD. Examples of the container include, but are not limited to, vials, tubes, and the like.

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### EXAMPLES

### Materials and Methods

### Cell Culture

Each type of cells used in the present study were grown in manufactures' suggested medium supplemented with 10% heat-inactivated fetal bovine serum, 100 units/mL penicillin, and 100 g/mL streptomycin, at 37° C. in a humidified 5% CO₂ incubator.

### IDUA Inhibition Assay

Compounds were mixed with recombinant human α-IDUA (rh-α-IDUA, Aldurazyme®, 1.2 nM as the final concentration) and the fluorogenic substrate, 4-methylberiilyl α-L-iduronic acid (4MU-IdoA; Cayman), at a final concentration of 100 μM in pH 3.5 buffer (50 mM NaOAc and 150 mM NaCl), then incubated in a 384-well microplate at 37° C. for 30 min. Stop solution (500 mM Na₂CO₃, pH 10.8) was added and fluorescence was read on a CLARIOstar® microplate reader (BMG LABTECH) at 355 nm excitation and 460 nm emission. Raw fluorescence counts were background subtracted, as defined by counts from substrate solution only. Inhibitory screening was performed and the active compounds were selected and further tested at lower concentration to determine their IC₅₀ values and Ki values.

### Example 1 Chemical Synthesis of Compounds of Formula (I)

The present compounds of formula (I) were prepared in accordance with procedures described in Schemes 1 to 5.

### 1.1 IdoA-Typed C-Glycoside 12

The synthetic journey started from the preparation of the intermediate 8 in multigram scale, synthesized from D-glucuronolactone (7) through the four-steps procedures described by Moffett (J Am Chem Soc 1954, 2813(12), 3310-3315). After C-glycosylation of 8 with allyltrimethyl silane, the anomeric isomers 9 and 10 were separated and the corresponding yield was 38% and 34%, respectively. Notably, the chirality and conformation of both were determined by NMR analysis and the results showed 9 possesses ⁴C₁ chair form but 10 remain ¹C₄ conformer. Next, the allyl group at the C-1 position of IdoA-typed C-glycoside 9 was transformed into the N-protected primary amine 11 through Lemieux-Johnson oxidation and reductive amination, followed by typical deprotection conditions to give CIdoA 12 (48% over four steps).

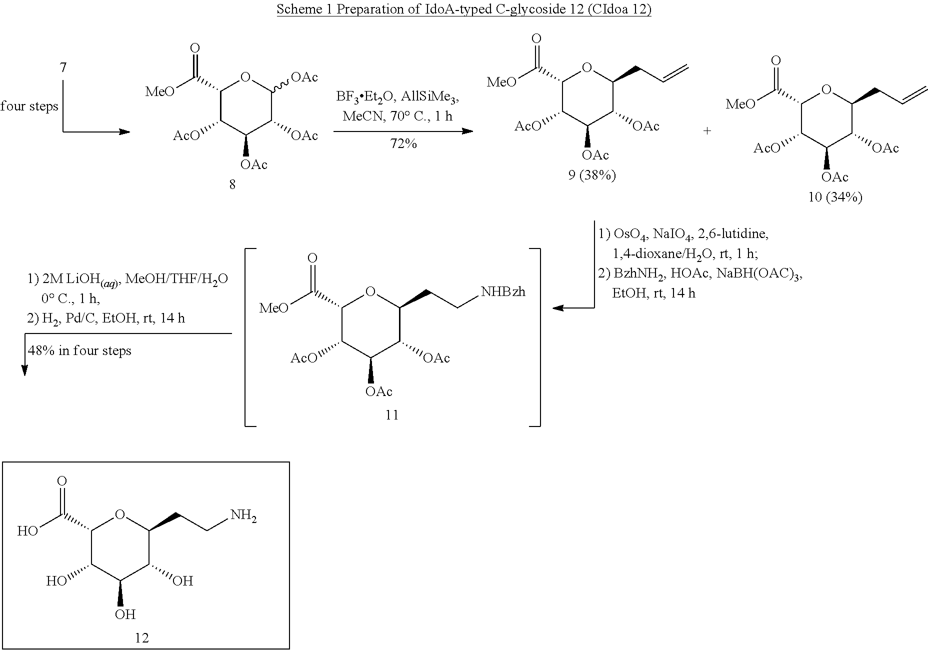

Compound 12. ¹H NMR (600 MHz, D₂O) δ 1.63-1.73 (m, 1H), 2.02-2.10 (m, 1H), 3.05 (ddd, J=13.2, 7.6, 6.0 Hz, 1H), 3.07-3.15 (m, 2H), 3.33 (dd, J=9.3 Hz, 1H), 3.33-3.40 (m, 1H), 3.57 (dd, J=9.6, 6.5 Hz, 1H), 4.23 (d, J=6.5 Hz, 1H). ¹³C NMR (150 MHz, D₂O) δ 176.4, 74.8, 73.7, 73.0, 71.9, 70.3, 36.8, 26.8. [C₈H₁₅NO₆+H]⁺ 222.0972, found 222.0968.

### 1.2 Diastereomers 15a and 15b

13 was prepared from commercially available D-glucose by following the procedures described by Tashiro (Bioorganic Med. Chem 2013, 21(11), 3066-3079). Then, 13 was condensed with benzylhydroxylamine to generate N-Benzyl-N-glycosylhydroxylamine 14 (87%). Subsequent deprotection of the C6 silane group of 13, followed by AllMgCl addition successfully generate the adduct in a multigram scale. Notably, the ratio of the unseparated adduct mixture was 1:3 based on the NMR analysis. The mixture of diastereomers was able to separate into 15a (minor, 15% yield) and 15b (major, 39% yield)

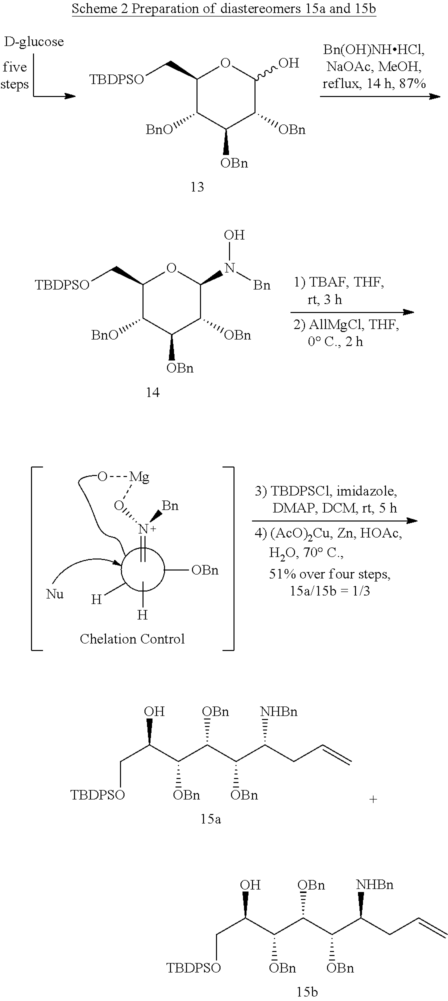

Alternatively, 15a and 15b were prepared in accordance with procedures described in Scheme 3, in which 13 was condensed with benzylamine to generate N-Benzyl-N-glycosylamine 18 (84%), subsequently diastereoselective addition of allylMgBr to 18 could generate 15a and 15b in the ratio of 5:1 after flash column chromatography.

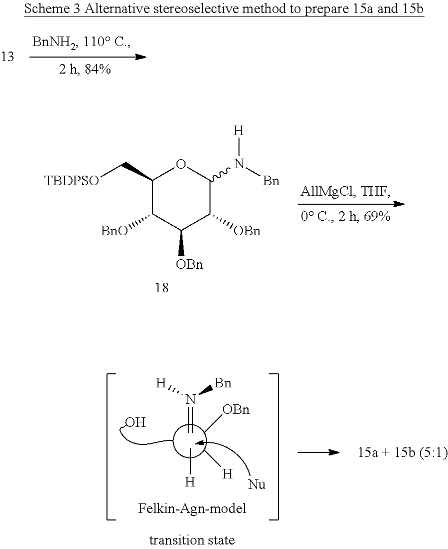

### 1.3 CIdoADNJ 23

15b was first subjected to N-Boc protection, followed by 0-protection with the methoxymethyl acetal group, and silane deprotection with TBAF to give 19 (76% over three steps). Then, sequential transformations including Swern oxidation, Pinnick oxidation, Steglich esterification, and N-Boc deprotection were performed; thus 19 was converted into 20 (42% over four steps). Next, mesylation of 20 with MsCl in the presence of activated powdered molecular sieves at 100° C. spontaneously gave the desired N-Benzyl protected iminosugar 21 with a methyl ester group at the C-6 position in the yield of 66%. Following the similar transformations from 9 to CIdoA 12 in Scheme 2, CIdoADNJ 23 were prepared from 21 (39% over four steps).

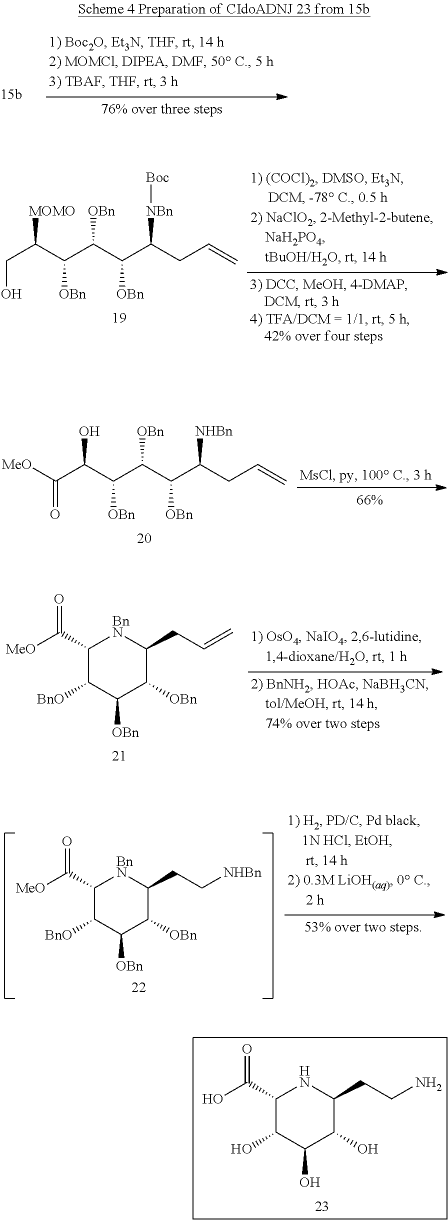

Compound 23. ¹H NMR (600 MHz, D₂O) δ 1.98-2.09 (m, 1H), 2.25-2.34 (m, 1H), 3.08-3.19 (m, 2H), 3.49 (dt, J=7.5, 7.0 Hz, 1H), 3.54 (dd, J=7.6 Hz, 1H), 3.60 (dd, J=7.4 Hz, 1H), 3.90 (dd, J=7.6, 4.7 Hz, 1H), 4.15 (d, J=4.7 Hz, 1H). 13C NMR (150 MHz, D₂O) δ 170.5, 72.4, 70.1, 68.3, 55.3, 54.6, 36.4, 27.3. HRMS calcd for [C₈H₁₆N₂O₅+H]⁺ 221.1132, found 221.1130.

### 1.4 CIdoA 27a-c and CIdoADNJ 28a-c

With the 12 and 23 in hand, the substituted reagents 24-26 bearing a negatively charged phosphate group were applied to conjugate to the primary amino moiety of 12 and 23 through an amide bond formation, followed by deprotection give the corresponding CIdoA 27a-c and CIdoADNJ 28a-c, respectively.

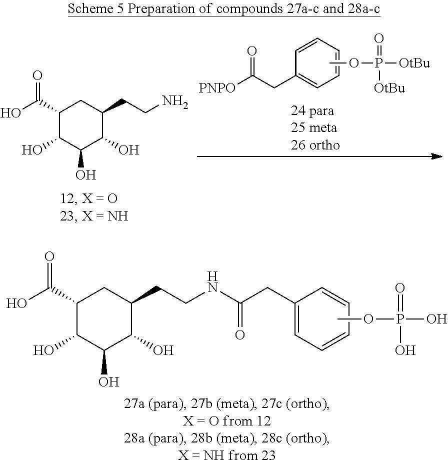

Compound 27a. ¹H NMR (600 MHz, D₂O) δ 1.5-1.6 (m, 1H), 1.9-2.0 (m, 1H), 3.1 (t, J=8.8 Hz, 1H), 3.1-3.2 (m, 1H), 3.2-3.3 (m, 1H), 3.5 (s, 2H), 3.6 (t, J=8.9 Hz, 1H), 3.6-3.7 (m, 2H), 4.4 (d, J=6.2 Hz, 1H), 7.1 (d, J=8.3 Hz, 2H), 7.2 (d, J=8.4 Hz, 2H). ¹³C NMR (151 MHz, D₂O) δ 174.5, 173.3, 163.0 (q, J=35.4 Hz), 151.1, 130.5, 130.4, 120.7, 120.7, 116.3 (q, J=291.7 Hz), 74.6, 73.7, 73.4, 72.6, 69.9, 41.7, 36.3, 29.9. ³¹P NMR (202 MHz, D₂O) δ −2.6. HRMS calcd for [C₁₆H₂₂NO₁₁P—H]⁻ 434.0858, found 434.0833.

Compound 27b. [α]_{D}²⁵ −46.4 (c 0.30, H₂O). ¹H NMR (600 MHz, D₂O) δ 1.5-1.6 (m, 1H), 1.9-2.0 (m, 1H), 3.1-3.2 (m, 2H), 3.3 (dt, J=14.1, 6.9 Hz, 1H), 3.5 (s, 2H), 3.6 (t, J=8.9 Hz, 1H), 3.6-3.7 (m, 2H), 4.4 (d, J=6.1 Hz, 1H), 7.0-7.0 (m, 2H), 7.0 (d, J=8.5 Hz, 1H), 7.3 (t, J=7.8 Hz, 1H). ¹³C NMR (151 MHz, D₂O) δ 174.1, 173.0, 162.9 (q, J=35.5 Hz), 152.0, 136.4, 130.1, 125.1, 121.3, 119.2, 116.2 (q, J=291.4 Hz), 74.6, 73.6, 73.2, 72.6, 69.8, 42.2, 36.4, 29.9. ³¹P NMR (202 MHz, D₂O) δ −2.8. HRMS calcd for [C₁₆H₂₂NO₁₁P—H]⁻ 434.0858, found 434.0837.

Compound 27c. ¹H NMR (600 MHz, D₂O) δ 1.5-1.6 (m, 1H), 1.9-1.9 (m, 1H), 3.1 (t, J=8.9 Hz, 1H), 3.2 (dt, J=13.1, 6.3 Hz, 1H), 3.3 (dt, J=13.6, 6.6 Hz, 1H), 3.6 (s, 2H), 3.6 (t, J=9.0 Hz, 1H), 3.6-3.7 (m, 2H), 4.3 (d, J=6.0 Hz, 1H), 7.0-7.1 (m, 1H), 7.2-7.3 (m, 3H). ¹³C NMR (151 MHz, D₂O) δ 174.1, 173.2, 163.0 (q, J=35.8 Hz), 150.5, 131.3, 128.9, 126.1, 124.3, 120.1, 116.3 (q, J=291.2 Hz), 74.7, 73.6, 73.3, 72.6, 70.0, 37.3, 36.4, 29.8. ³¹P NMR (202 MHz, D₂O) 6-2.8. HRMS calcd for [C₁₆H₂₂NO₁₁P—H]⁻ 434.0858, found 434.0833.

Compound 28a. ¹H NMR (600 MHz, D₂O) δ 1.8-1.9 (m, 1H), 2.1-2.2 (m, 1H), 3.2-3.3 (m, 2H), 3.4-3.5 (m, 1H), 3.5 (s, 2H), 3.6 (t, J=5.9 Hz, 1H), 3.8 (t, J=5.7 Hz, 1H), 4.1 (t, J=4.7 Hz, 1H), 4.3 (d, J=3.9 Hz, 1H), 7.1 (d, J=8.2 Hz, 2H), 7.2 (d, J=8.1 Hz, 2H). ¹³C NMR (151 MHz, D₂O) δ 175.5, 169.4, 162.9 (q, J=35.7 Hz), 151.0, 130.5, 130.4, 120.8, 120.7, 116.2 (q, J=291.4 Hz), 70.6, 69.1, 68.1, 55.1, 54.1, 41.5, 35.6, 28.2. ³¹P NMR (202 MHz, D₂O) 6-2.7. HRMS calcd for [C₁₆H₂₃N₂O₁₀P—H]⁻ 433.1018, found 433.0994.

Compound 28b. ¹H NMR (600 MHz, D₂O) δ 1.8-1.9 (m, 1H), 2.1-2.2 (m, 1H), 3.2-3.4 (m, 2H), 3.4-3.5 (m, 1H), 3.6 (t, J=5.8 Hz, 1H), 3.8 (t, J=5.8 Hz, 1H), 4.1 (t, J=4.9 Hz, 1H), 4.3 (d, J=3.7 Hz, 1H), 7.0-7.0 (m, 3H), 7.3 (t, J=8.2 Hz, 1H). 13C NMR (151 MHz, D₂O) δ 175.1, 169.4, 162.9 (q, J=35.2 Hz), 152.0, 136.3, 130.1, 125.1, 121.0, 116.2 (q, J=292.0 Hz), 70.6, 69.1, 68.1, 55.1, 54.2, 42.0, 35.6, 28.2. ³¹P NMR (202 MHz, D₂O) δ −2.6. HRMS calcd for [C₁₆H₂₃N₂O₁₀P—H]⁻ 433.1018, found 433.0995.

Compound 28c. Yield 31%. [α]_{D}²⁵ −17.5 (c 0.29, H₂O). ¹H NMR (600 MHz, D₂O) δ 1.9-1.9 (m, 1H), 2.1-2.2 (m, 1H), 3.3-3.3 (m, 1H), 3.3-3.3 (m, 1H), 3.5 (d, J=6.6 Hz, 1H), 3.6 (s, 1H), 3.7 (t, J=5.1 Hz, 1H), 3.8-3.8 (m, 1H), 4.1-4.1 (m, 1H), 4.3-4.3 (m, OH), 7.1-7.1 (m, 1H), 7.2-7.3 (m, 4H). ¹³C NMR (151 MHz, D₂O) δ 175.1, 166.9, 162.9, 150.4, 131.7, 129.0, 126.0, 124.4, 119.9, 117.3, 115.3, 70.7, 69.1, 68.2, 55.3, 54.1, 37.5, 35.6, 28.1. ³¹P NMR (202 MHz, D₂O) δ −2.9. HRMS calcd for [C₁₆H₂₃N₂O₁₀P—H]⁻ 433.1018, found 433.0993.

### Example 2 Characterization of the Compounds of Example 1

### 2.1 IDUA Inhibitory Assay

The compounds of Example 1 were evaluated on their inhibitory potential against rh-α-IDUA, and their apparent IC₅₀ values were determined by using the fluorogenic substrates 4-methylumbelliferyl-α-iduronide (4-MU-IdoA) and results are shown in Table 1.

CIdoA analogues 27a-c with IC₅₀ values in low-micromolar range proved more potent than CIdoADNJ analogues 28a-c, most of which were inactive at 250 μM except 28c. Satisfyingly, a significant reduction of IC₅₀ from 6.7 μM (12) to 1.0 μM (27c) indicated that the conjugation of negatively-charged phosphate group with certain orientation improved the inhibitory potency and then furnished 27c as the most potent IDUA inhibitors in the present disclosure. As to CIdoADNJ analogues, it was noted that 28c was superior than other CIdoADNJ analogues.

TABLE 31
Inhibitory activity of compounds 12,
27a-c and 23, 28a-c against rh-α-IDUA.

| Compound # | IC₅₀ (μM) | Compound # | IC₅₀ (μM) |
|---|---|---|---|
| 12 | 6.7 ± 0.3 | 23 | NI^{a} |
| | (Ki = 4.0 μM) | | |
| 27a | 5.4 ± 0.5 | 28a | NI^{a} |
| 27b | 4.0 ± 0.5 | 28b | NI^{a} |
| 27c | 1.0 ± 0.06 | 28c | 43%^{b} |

^{a}NI refers to less than 10% inhibition at 250 μM.
^{b}Inhibition % at 250 μM.

### 2.2 α-IDUA Stabilizers

In this example, the potential of the compound of Example 1 acting as an α-IDUA protein stabilizer was investigated in two MPSI cell lines—GM01391 and GM02846, which possessed insufficient residual IDUA enzyme activity.

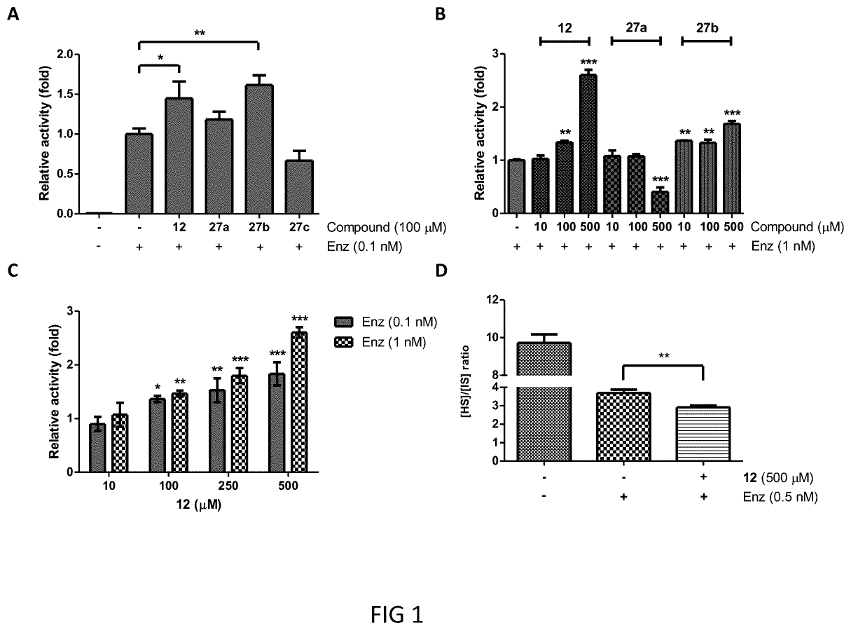

To this purpose, GM01391 fibroblasts were treated with a combination of rh-α-IDUA (0.1 nM) and compounds 12, and 27a-c (100 aM); or with rh-α-IDUA for 1 day, followed by a 1-day wash-out. To measure the intracellular IDUA activity, the corresponding cells were harvested and lysed before the incubation with the fluorogenic substrate. Results are illustrated in FIG. 1.

Treatment with rh-α-IDUA dramatically increased the IDUA activity due to the severely insufficient residual IDUA in the cells, and the results showed that 12 and 27b enabled 1.4 and 1.6-fold enhancement of IDUA activity rather than the treatment of rh-α-IDUA alone (FIG. 1, (A)). On the other hand, a moderate reduction of IDUA activity was observed in the 27c co-treated cells, presumably due to the potent inhibition of 27c against rh-α-IDUA. Also, IDUA activity was barely improved by the co-treatment with 27a.

To confirm CIdoA analogues 12, 27a or 27b as potential IDUA stabilizer, dose-dependence experiments were performed, and the results indicated that IDUA activity was nearly three times higher in GM01391 fibroblasts with the combined treatment of rh-α-IDUA (1 nM) and 12 (500 μM) than those treated with rh-α-IDUA rh-α-IDUA (1.7-fold enhancement at 500 μM), and the IDUA activity was even suppressed by the co-treatment of 27a at 500 μM (FIG. 1, (B)).

Further dose-dependence investigation of the 12/rh-α-IDUA co-treatment was performed. The IDUA activity of GM01391 fibroblasts was enhanced by the 12/rh-α-IDUA co-treatment in a dose-dependent manner compared to rh-α-IDUA treatment alone, revealing that 12 could stabilize rh-α-IDUA and improved the cellular uptake of rh-α-IDUA (FIG. 1, (C)). Further, the dose-response of 12 became more significant when the concentration of rh-α-IDUA was raised to 1 nM.

The time course of 12/rh-α-IDUA co-treatment showed that the enhancement of IDUA activity by 12 could be maintained even after 1-day treatment followed by 8-day wash-out (data not shown).

Generally, accumulation of GAGs could be observed in MPS patients due to the dysfunction of any lysosomal enzymes involved in the degradation of GAGs including IDUA associated with MPSI, which means the enhanced efficacy of protein drugs by the co-treatment with protein stabilizers could be evaluated more directly by the improved reduction of GAGs than the increased activity of MPS-related lysosomal enzymes. As a result, a robust method for quantitative detection of HS, one of the types of GAGs excessively stored in MPS I patients, was established by us for the evaluation of potential therapeutic effect of compound 12 in MPSI.

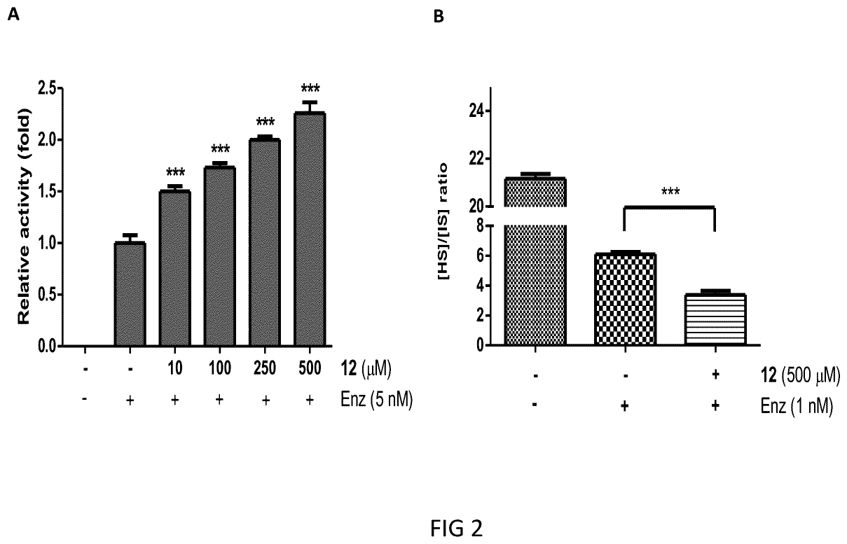

HS of the in situ treated fibroblasts were extracted and chemically cleaved to generate HS disaccharides as the released biomarker, which could be quantitatively measured via LC-MS/MS with a synthesized novel disaccharide by us as the internal standard. With this established method for cell-based HS analysis, the qualified CIdoA analogue 12 was further evaluated. A statistically significantly improved reduction of the accumulated substrate was shown in the 12/rh-α-IDUA co-treated GM01391 fibroblasts by the stabilization effect of 12 toward rh-α-IDUA than the rh-α-IDUA treatment alone (FIG. 1, (D)). Similar results were also found in 12/rh-α-IDUA co-treated GM02846 fibroblasts, in which 12 dose-dependently enhanced rh-α-IDUA activity (FIG. 2, (A)), and a combination of 12 and rh-α-IDUA also led to significant reduction of HS than the treatment of rh-α-IDUA alone (FIG. 2, (B)).

Collectively, 12 was proved to be a IDUA stabilizer with potential therapeutic effect in MPSI by the cell-based IDUA activity assay and quantitative analysis of HS in GM01391 and GM02846 fibroblasts.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of the present disclosure.

What is claimed is:

1. A compound of formula (I), or a pharmaceutically acceptable salt, a solvate or a stereoisomer thereof, 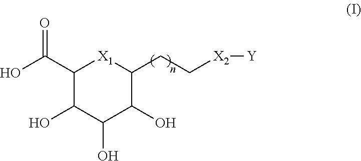
wherein,
n is an integral between 0 and 2;
X₁ is O or —NH;
X₂ is O, —NH or —NR^{a}, in which R^{a} is C₁₋₁₀ alkyl; 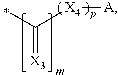
Y is H or
in which
m and p are independently 0 or 1;
X₃ is S, O, or —NH;
X₄ is O, —NH, methylene, or —CH₂(C₁₋₁₀)alkyl; and
A is aryl, heteroaryl, or heterocyclyl optionally substituted with one or more substituent selected from the group consisting of halo, hydroxyl, amino and phosphate.

2. The compound of claim 1, wherein in the formula (I), n is 1, X₁ is O, X₂ is —NH, and Y is H.

3. The compound of claim 1, wherein the compound has the structure of formula (II), 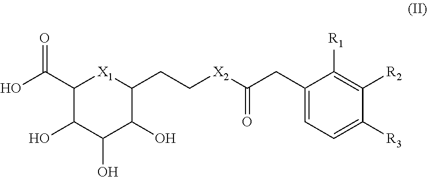
wherein,
X₁ is O or —NH;
X₂ is O, —NH or —NR^{a}, in which R^{a} is C₁₋₁₀alkyl; and
R₁, R₂, and R₃ are independently H or phosphate.

4. The compound of claim 3, wherein the compound is selected from the group consisting of,

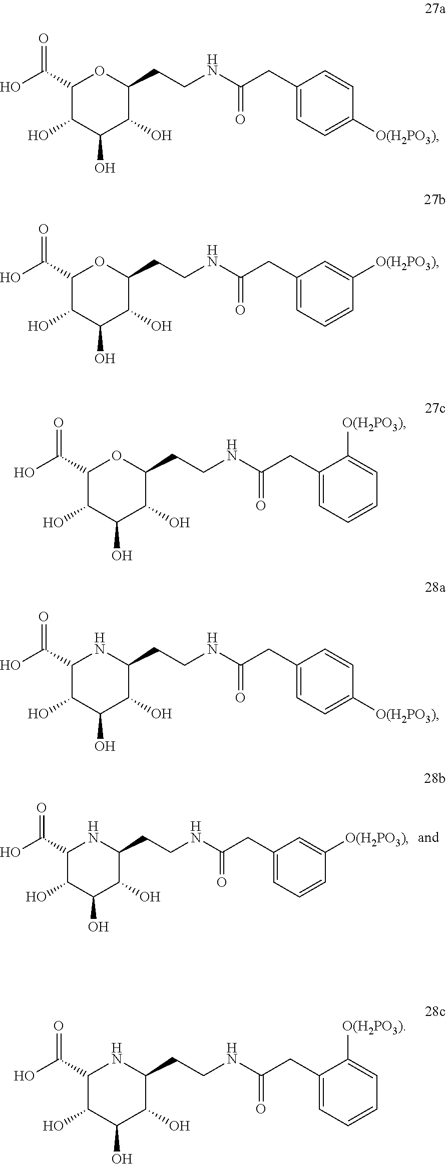

5. The compound of claim 4, wherein the compound is

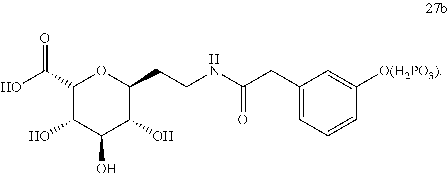

6. A method for treating mucopolysaccharidosis type I (MPSI) in a subject comprising administering to the subject a therapeutically effective amount of a recombinant human α-Iduronidas (rh-α-IDUA) together with, before or after the administration of the compound of claim 1.

7. The method of claim 6, wherein in the formula (I), n is 1, X₁ is O, X₂ is —NH, and Y is H.

8. The method of claim 6, wherein the compound has the structure of formula (II), 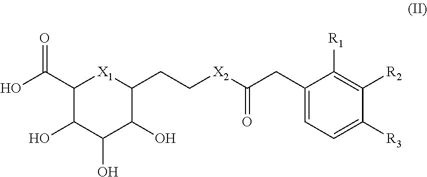
in which,
X₁ is O or —NH;
X₂ is O, —NH or —NR^{a}, in which R^{a} is C₁₋₁₀alkyl; and
R₁, R₂, and R₃ are independently H or phosphate.

9. The method of claim 8, wherein the compound is selected from the group consisting of,

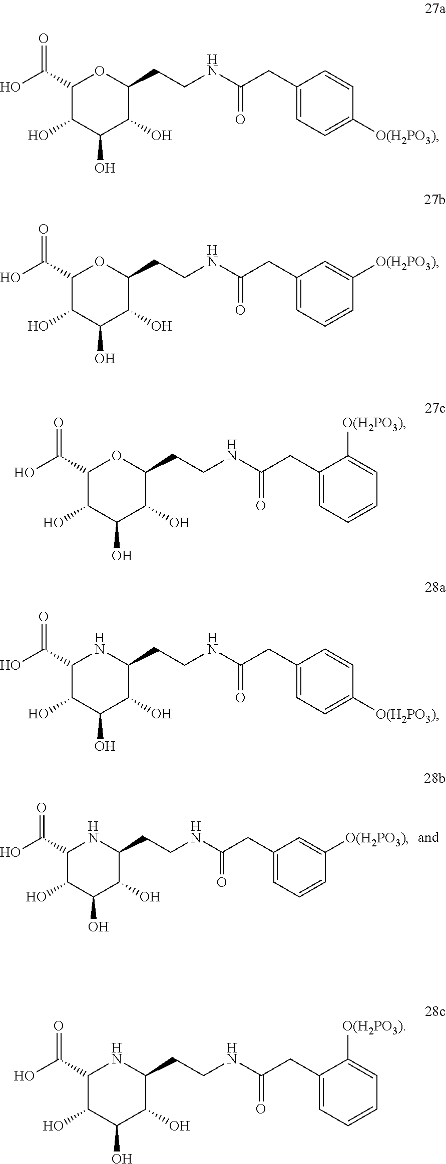

10. The method of claim 8, wherein the compound is

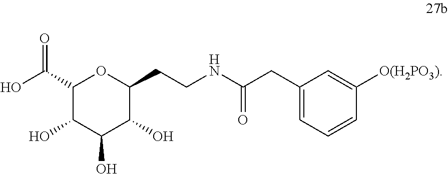

11. The method of claim 6, wherein the subject is a human.
